(19) United States
(12) Patent Application Publication
(10) Pub. No.: US 2018/0303341 A1
(43) Pub. Date: Oct. 25, 2018

(54) OPTICAL INSTRUMENT

(71) Applicant: Cats Tonometer, LLC, Tucson, AZ (US)

(72) Inventor: Sean J. McCafferty, Tucson, AZ (US)

(21) Appl. No.: 15/565,244

(22) PCT Filed: Aug. 27, 2015
(86) PCT No.: PCT/US2015/047134

§ 371 Date: Oct. 9, 2017

Related U.S. Application Data
(60) Provisional application No. 62/148,048, filed on Apr. 15, 2015.

(51) Int. Cl. A61B 3/16; A61B 5/00; A61B 5/03

(52) U.S. Cl. CPC A61B 3/16; A61B 2560/0406; A61B 5/03; A61B 5/0082

(57) ABSTRACT

Ophthalmological device including an applanation tonometer tip having a bi-curved cornea-contacting surface structured to minimize the intracorneal stress, and method of using such device for measurement of intraocular pressure. The cornea-contacting surface includes a first central portion and a second portion that encircles and adjoins the first central portion. The curvatures of the first and second portions have opposite signs. In one case, the first central portion can be rotationally-symmetric. In a related case, the first portion has a curvature with a sign opposite to that of a curvature of a typical cornea, while the curvature of the second portion has a sign equal to that of the curvature of the cornea. Method for using the device to procure values IOP with increased ac curacy as compared with the use of a conventional flat-surface tonometer tip.

### CROSS-REFERENCE TO RELATED APPLICATIONS

The present application claims priority and benefit from the U.S. Provisional Patent Application No. 62/148,048, filed on Apr. 15, 2015 and titled “Ophthalmological Instrument”. The present application is a continuation-in-part of U.S. patent application Ser. No. 14/012,592 now published as U.S. 2014/0073897. The disclosure of each of the above-referenced applications is incorporated herein by reference.

### TECHNICAL FIELD

The present invention relates to an ophthalmological instrument and, more particularly, to a tip for applanation tonometer that is structured as a cornea-contacting member and the applanation tonometer utilizing such tip.

The conventionally used Goldmann applanation tonometer (presented schematically in FIG. 1B and discussed further below) utilizes a flat, planar surface tip (the tip a cornea-contacting surface of which has a zero curvature). The use of which is known to inevitably require a correction (of the results of the measurements of the intraocular pressure in the eye) to account for non-zero corneal thickness and stiffness. It is also well recognized that the accuracy of such correction is often questionable, as the correction is predicated on the unpredictable degree of correlation between the stiffness and thickness of the cornea. There remains a need in a tonometer tip the use of which would allow to alleviate—if not remove completely—the need for correcting the results of the measurements of the intraocular pressure

### SUMMARY

The idea of the invention stems from the realization that the above-mentioned drawback of the conventionally-used Goldmann applanation tonometer is caused, in significant part, by the flatly-shaped tonometer tip. Moreover, a cause of yet another error in the measurement of the intraocular pressure (IOP)—neither compensated by the existing flat tonometer tip nor addressed by the related art—is the contribution of the non-zero curvature of the cornea. As explained in more detail below, the difference between the curvatures of the flat tonometer tip (zero curvature) and a non-zero curvature cornea cases a ripple or kink in the surface of cornea during the applanation procedure, which significantly distorts the corneal surface, causing intracorneal stress that, in turn, adds errors to the measurement of the IOP. On the other hand, the cornea with non-zero curvature forms a component of force transferred to the tonometer tip and even further obscuring IOP measurement.

False measurement of the IOP with the existing tonometer tip (the exact amount of required corrections for which remains very uncertain—creates a risk for misdiagnosis and/or delayed detection of ophthalmological diseases.

These drawbacks of the conventional measurement of the IOP with the use of a tonometer are resolved by contraptions of the present invention. In particular, a persisting problem of the need for a largely-undefined correction of the results of an IOP measurement performed with an applanation tonometer is solved by providing a tonometer with a tip the corneal contact surface of which is judiciously curved and not flat. Equipping the tonometer tip's surface with a curvature as discussed reduces and, in some cases, eliminates measurement errors caused by corneal curvature and intracorneal stress, thereby allowing a user to rely on raw results of direct IOP measurement carried out with the tonometer tip of the invention.

Embodiments of the invention provide an optical instrument for measurement of intraocular pressure (IOP) of an eye. Such instrument includes at least a corneal contact member having a longitudinal axis (referred to herein as “axis”) along which the corneal contact member may be moved in operation, axis, and a front surface that is dimensioned to contact the cornea of an eye during the measurement. The longitudinal axis of the corneal contact member is preferably an axis of symmetry of the corneal contact member. The front surface includes at least a) corneal contact surface portion, which portion defines a central portion of the front surface of the corneal contact member and which portion is being curved to reduce an error contributed to said measurement by at least a curvature of the cornea; and b) a peripheral surface portion surrounding a curved corneal contact surface portion and tangentially merging with said corneal contact surface portion along a closed plane curve.

In one example, where the cornea of the eye has a first curvature having a first sign, the corneal contact surface portion has a second curvature with a second sign opposite to the first sign, while the peripheral surface portion has a third curvature with a third sign (such third sign being opposite to the second sign). In such specific example, the front surface may be shaped to change a sign of the first curvature within a surface area defined by an area of contact between said corneal contact member pressed against the cornea and the cornea. In a related alternative example, the cornea of the eye has a first curvature having a first sign, and the curved corneal contact surface portion has a second curvature with a second sign that is equal to the first sign. Here, the peripheral surface portion has a third curvature with a third sign (the third sign being opposite to the second sign). In any example, the front surface may be shaped and dimensioned to flatten a portion of the cornea when said corneal contact member is pressed, in operation, against the cornea, the flattened portion of the cornea defined by a surface area being preferably symmetric about the axis, thereby simplifying the measurement of the IOP.

The optical instrument may additionally include an optical prism in a body of the corneal contact member, and a source of light positioned to transmit light through the prism towards the front surface. Alternatively or in addition, the corneal contact surface portion may be configured to define a portion of a spherical surface. Alternatively or in addition, the front surface may be configured to be axially symmetric about the axis and, in a specific case, the optical instrument is configured as a tonometer. The instrument may be additionally equipped with a housing element having an outer conical surface such that the corneal contact member is fixed in the housing element.

Embodiments of the invention also provide an optical instrument, for measurement of intraocular pressure (IOP), that includes a corneal contact member having a front surface that is dimensioned to contact a first portion of the cornea of an eye, for example, as explained above. Preferably, the front surface is rotationally symmetric about an axis. Such front surface may contain at least (i) a corneal contact surface portion defining a portion of a spherical surface devoid of openings therethrough, where the corneal contact surface portion has a first curvature with a first sign opposite to a sign of a corneal curvature; and (ii) a peripheral surface portion surrounding the corneal contact surface portion and tangentially merging with the corneal contact surface portion along a closed curve defined in a plane that is transverse to said axis, the peripheral surface portion having a second curvature, the second curvature having a second sign that is equal to a sign of a curvature of the cornea. In a specific implementation of the instrument, the front surface may be shaped to applanate a portion of the cornea when the corneal contact member is pressed, in operation, against the cornea, the applanated portion of the cornea defined by an annulus. In such specific implementation, the front surface is dimensioned to minimize intracorneal stress in said applanated portion of the cornea. Alternatively or in addition, the optical instrument may include an optical prism in a body of the corneal contact member and a source of light positioned to transmit light through the prism towards the front surface.

Embodiments of the invention additionally provide a method for measuring intraocular pressure (IOP) with an optical instrument the examples of structure of which are discussed in more detail with respect to the Drawings. The instrument used for measuring the IOP may include a corneal contact member (having a corneal contact surface that defines at least one of (i) a central curved portion having a surface curvature of a first sign, and (ii) a peripheral surface portion having a surface curvature of a second sign, where peripheral surface portion surrounding the central curved portion). The method includes at least one of the steps of (i) pressing the corneal contact member against the cornea to establish a contact between the corneal contact surface and the cornea and to applanate a first portion of the cornea while minimizing an error contributed to said measuring by a curvature of the cornea; (ii) forming an optical image of the cornea in light traversing the corneal contact member and the corneal contact; and (iii) determining a value of the IOP from imaging data representing the optical image. The steps of pressing may include pressing the central curved portion against the cornea while curvatures of the central curved portion and the cornea have opposite signs. (Optionally, the step of pressing is effectuated when curvatures of the central curved portion and the peripheral surface portion have opposite signs.) In one implementation, the method is devoid of a step of correction of the imaging data to compensate for at least one of the corneal thickness and stiffness. Furthermore, the step of pressing may include pressing the corneal contact member in which the peripheral surface portion is tangentially merging with the central curved portion along a closed plane curve.

### BRIEF DESCRIPTION OF THE DRAWINGS

The invention will be more fully understood by referring to the following Detailed Description in conjunction with the generally not-to-scale Drawings, of which:

FIG. 1A presents two views of Goldmann applanation tonometer tip used for measurements of a human eye, showing the bi-prism angle (60 degrees);

FIG. 1B is a diagram illustrating a Goldmann applanation tonometer;

FIG. 2A is a diagram illustrating flattening of the corneal surface due to pressure applied by the tonometer tip;

FIG. 2B is a diagram showing the pressure-dependent positioning of two semi-circles representing an image of the flattened portion of the corneal surface;

FIGS. 3A and 3B are cross-sectional and top views that illustrate schematically a tonometer tip according to an embodiment of the invention;

FIG. 3C is a diagram illustrating an alternative embodiment of the invention;

FIG. 4 is a diagram illustrating a method for measurement of intraocular pressure with an embodiment of FIGS. 3A, 3B;

FIGS. 5A and 5B are cross-sectional and top views that illustrate schematically a tonometer tip according to an alternative embodiment of the invention;

FIG. 6 illustrates a specific embodiment of a surface of the tonometer tip;

FIG. 7 illustrates von Misses stress in a standard cornea caused by a measurement of the IOP with the embodiment of FIGS. 5A, 5B;

FIG. 8 provides plots illustrating surface profiles of a corneal surface before and after applanation with the embodiment of FIGS. 5A, 5B;

FIG. 9A provides plots illustrating errors caused by the corneal curvature during the measurement of the IOP with a flat-tip tonometer piece, the embodiment of FIGS. 3A, 3B and the embodiment of FIGS. 5A, 5B;

FIG. 9B provides plots illustrating errors caused by the corneal rigidity during the measurement of the IOP with a flat-tip tonometer piece and the embodiment of FIGS. 5A, 5B;

FIG. 9C provides plots illustrating errors caused by non-zero corneal thickness during the measurement of the IOP with a flat-tip tonometer piece and the embodiment of FIGS. 5A, 5B;

FIG. 10 is a contour plot showing isobaric curves as a function of the corneal thickness for a standard cornea;

FIGS. 11A and 11B provide specific cross-sectional profiles for embodiments of FIGS. 3A and 5A, respectively;

FIG. 12 is a plot showing the reduction of average stress in cornea applanated with a flat-tip tonometer piece, the embodiment of FIGS. 3A, 3B and the embodiment of FIGS. 5A, 5B

### DETAILED DESCRIPTION

The discussed invention solves problems accompanying the measurements of intraocular pressure in the eye that are conventionally performed with the use of a Goldmann-type applanation tonometer (GAT) having a flat tip. The invention further facilitates such measurements by removing the need to correct the results of the measurements for the contribution of corneal thickness and stiffness, while at the same time minimizing both the error of the IOP-measurement caused by the corneal curvature, corneal rigidity, and the intraocular stress imposed on the eye-ball my the measurement procedure but ignored clinically to-date. Such advantageous effects are achieved by employing a tonometer tip having the cornea-contacting (generally axially symmetric) surface configured to include at least i) a central curved portion and ii) a peripheral portion encircling the central portion having a curvature with a sign opposite to the sign of the curvature of the central portion. The central and peripheral portions of the tonometer tip surface may merge tangentially along a closed plane curve. Counter intuitively—and to a noticeable advantage (over the conventional design of a tonometer member having a tip with a flat, not curved surface) in terms of minimization of intracorneal stress during the measurement—the curvature of the central portion of the surface of the tip of one specific embodiment preferably has a sign opposite to that of the curvature of the cornea. In accordance with embodiments of the present invention, methods and apparatus are disclosed for an ophthalmological instrument including a corneal contact member structured according to the idea of the invention for use with the GAT platform. Embodiments of the invention include a tonometer tip, containing a biprism-containing portion and a corneal contact surface the shape of which that is configured to minimize deformation of the corneal surface and the intracorneal stress during measurement of the intraocular pressure.

For the purposes of this disclosure and the appended claims, and unless stated otherwise:
A plane curve is a curve defined in a plane. A closed plane curve is a curve with no end points and which completely encloses an area. Preferably, the closed plane curve is defined in a plane that is transverse to the axis, that is in a plane that is lying or extending across (or in a cross direction) with respect to the axis and in a specific case—in a plane that extends orthogonally to the axis. This enhances an homogeneity of deformation of the cornea when the corneal contact surface portion of the corneal contact member is being pressed against the cornea.
Generally, a surface of the corneal contact member has a surface that deviates from a flat surface and that includes two surface portions curved differently, one being a concave surface portion and another being a convex surface portion. For the purposes of this disclosure and appended claims, terms such as radius of curvature, curvature, sign of curvature and related terms are identified according to their mathematical meanings recognized and commonly used in related art. For example, a radius of curvature of a given curve at a point at the surface is defined, generally, as a radius of a circle that most nearly approximates the curve at such point. The term curvature refers to the reciprocal of the radius of curvature. A definition of a curvature may be extended to allow the curvature to talk on positive or negative values (values with a positive or negative sign). This is done by choosing a unit normal vector along the curve, and assigning the curvature of the curve a positive sign if the curve is turning toward the chosen normal or a negative sign if it is turning away from it. For the purposes of the present disclosure and the accompanying claims, a sign of a given curvature is defined according to such convention. For definitions of these and other mathematical terms, a reader is further referred to a standard reference text on mathematics such as, for example, I. N. Bronstein, K. A. Semendyaev, Reference on Mathematics for Engineers and University Students, Science, 1981 (or any other edition).
References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention. Within this specification, embodiments have been described in a way that enables a clear and concise specification to bet written, but it is intended and will be appreciated that embodiments may be variously combined or separated without parting from the scope of the invention. In particular, it will be appreciated that all features described herein at applicable to all aspects of the invention.
When the present disclosure describes features of the invention with reference to corresponding drawings (in which like numbers represent the same or similar elements, wherever possible), the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.
Moreover, if the schematic flow chart diagram is included, the depicted order and labeled steps of the logical flow are indicative of one embodiment of the presented method. Other steps and order of steps may be conceived that are equivalent in function, logic, or effect to one or more steps, or portions thereof, of the illustrated method. Without loss of generality, the order in which processing steps or particular methods occur may or may not strictly adhere to the order of the corresponding steps shown.
The invention as recited in claims appended to this disclosure is intended to be assessed in light of the disclosure as a whole, including features disclosed in prior art to which reference is made.
For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. The use of this term in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated may vary within a range defined by an experimental measurement error that is typical when using a measurement method accepted in the art for such purposes. For example, a reference to a vector or line or plane being substantially parallel to a reference line or plane is to be construed as such vector or line extending along a direction or axis that is the same as or very close to that of the reference line or plane (with angular deviations from the reference direction or axis that are considered to be practically typical in the art, for example between zero and fifteen degrees, more preferably between zero and ten degrees, even more preferably between zero and 5 degrees, and most preferably between zero and 2 degrees). A term “substantially-rigid”, when used in reference to a housing or structural element providing mechanical support for a contraption in question, generally identifies the structural element that rigidity of which is higher than that of the contraption that such structural element supports. As another example, the use of the term “substantially flat” in reference to the specified surface implies that such surface may possess a degree of non-flatness and/or roughness that is sized and expressed as commonly understood by a skilled artisan in the specific situation at hand. For example, the terms “approximately” and about”, when used in reference to a numerical value, represent a range of plus or minus 20% with respect to the specified value, more preferably plus of, minus 10%, even more preferably plus or minus 5%, most preferably plus or minus 2%.
The term “surface” is used according to its technical and scientific meaning to denote a boundary between two media or bounds or spatial limits of a tangible element; it is understood as that which has length and breadth but not thickness, a skin (with a thickness of zero) of a body.
The terms “applanation”, “applanate”, “flattening”, “flatten” and the like generally refer to a process of action as a result of which a surface curvature of a subject at hand is being reduced, that is, the surface is being flattened or applanated (resulting in a surface that is either completely flat or a curvature of which is at least reduced as compared to the initial value of curvature).
In addition, the following disclosure may describe features of the invention with reference to corresponding drawings, in which like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

General Considerations.

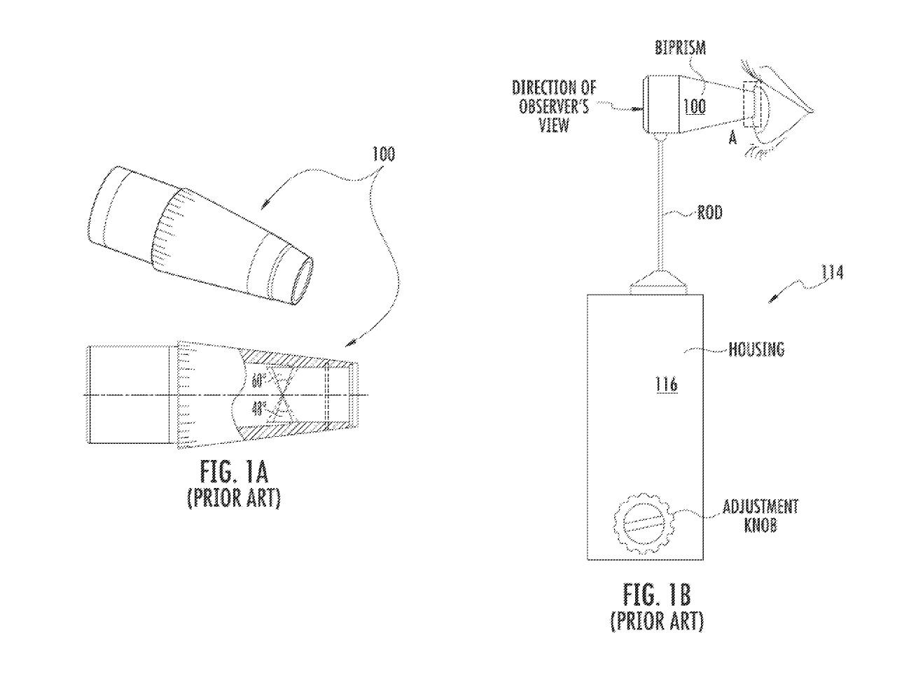

Tonometry is a non-invasive procedure that eye-care professionals perform to determine the intraocular pressure (IOP), the fluid pressure inside the eye. It is an important test in the evaluation of patients at risk from glaucoma, a disease often causing visual impairment in a patient. In applanation tonometry the intraocular pressure is inferred from the force required to flatten (applanate) a constant, pre-defined area of the cornea, as per the Imbert-Fick hypothesis that holds that when a flat surface is pressed against a closed sphere with a given internal pressure, an equilibrium will be attained when the force exerted against the spherical surface is balanced by the internal pressure of the sphere applied over the area of contact. In other words, pressure P within a flexible, elastic (and presumably infinitely thin) sphere is approximately equal to the external force f required to flatten a portion of the sphere and normalized by an area A that is flattened, P=f/A. Accordingly, a transparent pressure member with a planar contact surface (such as the element 100 as shown in FIG. 1A, for example) is pressed against the cornea of an eye in such a way that the latter is flattened over a pre-determined area (that in practice is about 7.3 mm²).

Before performing the measurement, and because the pressure member makes contact with the cornea, a topical anesthetic (such as proxymetacaine) is typically introduced on to the surface of the eye (for instance, in the form of eye drops). During the measurement, the eye is illuminated by blue light (for example, light delivered from a lamp equipped with a blue filter). In the zone of contact between the surface of the cornea and the pressure member, the film of tears (which contains fluorescein and has green-yellowish hue when illuminated with the blue light) is displaced, as a result of the contact, so that the boundary between the flattened and the curved areas of the cornea is readily identifiable. The contact pressure required for flattening is used as a measure of intraocular pressure.

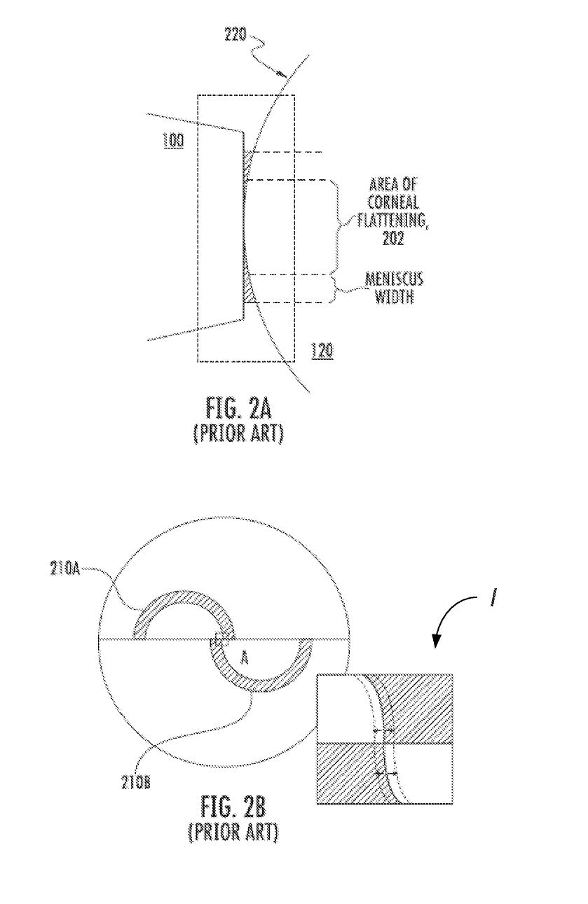

The classical Goldmann tonometer (see an example 114 in FIG. 1B) has a transparent plastic applanating tip 100 shaped as a truncated cone (with a flat surface that is brought in contact with the cornea in operation of the tonometer). The surface of cornea 120 is observed through the plastic applanation tip with the slit-lamp microscope. This device is the most widely used version of the tonometer in current practice of tonometry that utilizes the applanation of the cornea 120. The tip 100 (also referred to as a pressure member, or a corneal contact member) typically contains a bi-prism (a combination of two prisms touching at their apices), which, in reference to FIG. 2A, produces optical doubling of the image of the flattened surface 202 and separates the two image components by a fixed distance or space, across the field of view, which distance or space is dependent on the apex angles of the prisms. In further reference to FIG. 1B, the Goldmann tonometer corneal contact member or tip 100 is connected by a lever arm to the tonometer body 116. The tonometer body 116 contains a weight that can be varied.

The observer-examiner uses an optical filter (usually, a cobalt blue filter) to view the two image components (shown as semicircles 210A, 210B in FIG. 2B) formed through the applanating tip 100. The force applied through the tonometer tip 100 to the surface 220 of the cornea 120 is then adjusted using a dial (knob) connected to a variable tension spring of the device until the inner edges of the semicircles 210A, 210B, viewed in the viewfinder, are made to meet or coincide (see insert I of FIG. 2B). Such “meeting of the edges” occurs when a corneal area of about 3.06 mm in diameter has been flattened and when the two opposing, counteracting forces (the first produced by the resistance of the rigid cornea and the second produced by the tension of the tear film) become substantially equal and cancel each other out, thereby allowing the pressure in the eye to be determined from the force applied to the cornea. A non-invasive method, this method of determining an intraocular pressure is inherently imprecise.

Some of the measurement errors arise due to the fact that a cornea, unlike the ideal sphere, has non-zero thickness: a thinner than average cornea typically results in an underestimation of the IOP, while a thicker than average cornea may result in an overestimate of the actual IOP. To counterbalance the non-zero stiffness of the cornea and in order to applanate a portion of the cornea, additional force is required that cannot be counted towards the actual value of IOP. The studies revealed a correlation between the corneal thickness and corneal stiffness. Clearly, then, the non-zero thickness and stiffness of the cornea introduce the errors to the measurements of the IOP. Accordingly, to reduce-the IOP-measurement error, the value of the force applied to the cornea as measured initially has to be corrected in reference to a second measurement of corneal thickness (the latter measurement being performed using a pachymeter). The accuracy of such correction is predicated upon the accuracy of correlation between the thickness and stiffness characteristics of the cornea, which is also inherently inaccurate (due to influence of such variable factors as age of the person, a diameter of the cornea, corneal curvature, and effects produced by various eye diseases).

Additional cause of the measurement error—not addressed to-date in the art—is the contribution of the non-zero corneal curvature. It was theorized that the influence of the corneal curvature on the accuracy of the IOP measurement may be explained by the difference in the volume of the displaced eye-fluid after the area of the cornea is flattened, and/or the difference in the original volume of the eye, or both (Liu and Roberts, Influence of corneal biomechanical properties on intraocular pressure measurement, J. Cataract Refract. Surg., vol. 31, pp. 146-155, January 2005). The effect of the corneal curvature is independent from the intraocular pressure but manifests an important component of the force transferred from the eye-ball to the tonometer tip, with which it is in contact.

Finally, by the very fact of “flattening” of a portion of the otherwise non-flat cornea with which the conventional, flat-tip tonometer prism is brought in contact, the conventional “cornea-applanating” procedure of measuring the IOP produces a sort-of “kink” at a corneal surface. This “kink” manifests a corneal area, in which the curvature of the partially-applanated cornea is changing at a very high rate. This “kink” area, understandably, lies in the vicinity of a perimeter of the applanated portion of the cornea and defines the spatial transition between such applanated portion and the still-curved portion of the cornea that is not in contact with the flat tip of the tonometer. Phrased differently, at the “kink” area the value of the second derivative of the function representing the shape of the partially-applanated cornea is very high and the cornea is significantly distorted, which leads to intracorneal stress (causing additional component of fore and pressure applied to the tonometer tip, which component is not related to the IOP and adds an error to the measurement thereof).

Notably, to-date there is no conclusive and consistent data on the magnitude of corneal biomechanical properties. False IOP readings—the exact amount of required corrections for which remain uncertain—create the risk for misdiagnosis, resulting in missed or delayed detection of ophthalmological diseases. Therefore, a measurement technique and system that increase the precision and accuracy of the IOP results are required. The use of embodiments of the present invention increases the accuracy of the measurement of the IOP (performed, for example, with the use of a Goldmann applanation tonometer), thereby eliminating a need in an auxiliary measurement of the corneal thickness and reducing the overall cost of the IOP measurement and increasing the quality of care. Moreover, the use of embodiments of the invention minimizes both the contribution of the corneal curvature to the IOP-measurement procedure and the intraocular stress caused by such procedure on the eye.

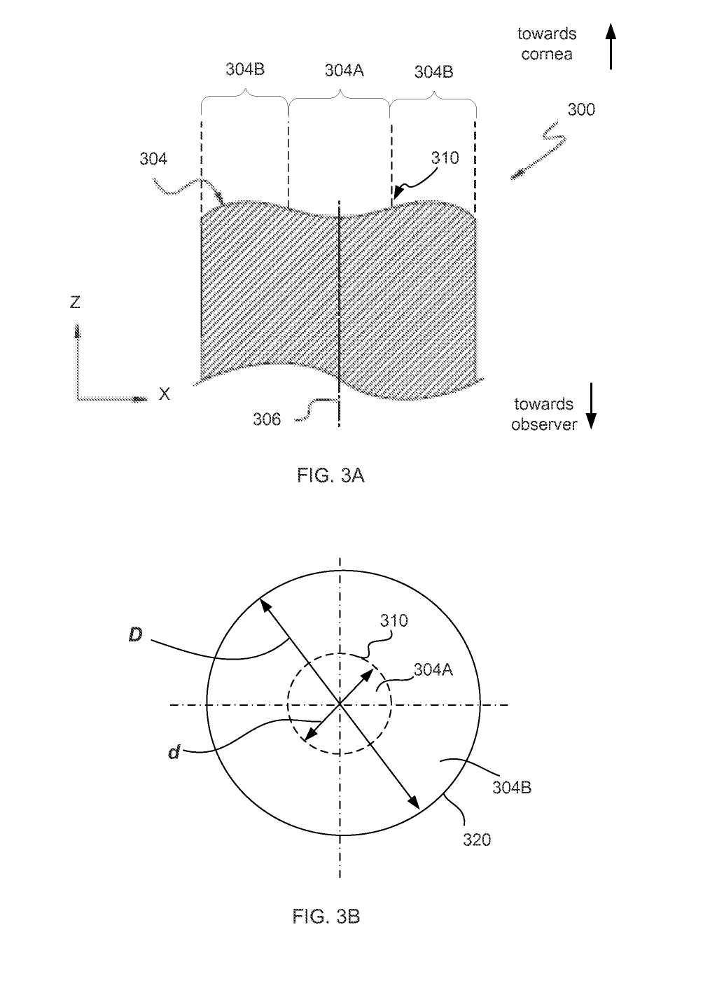

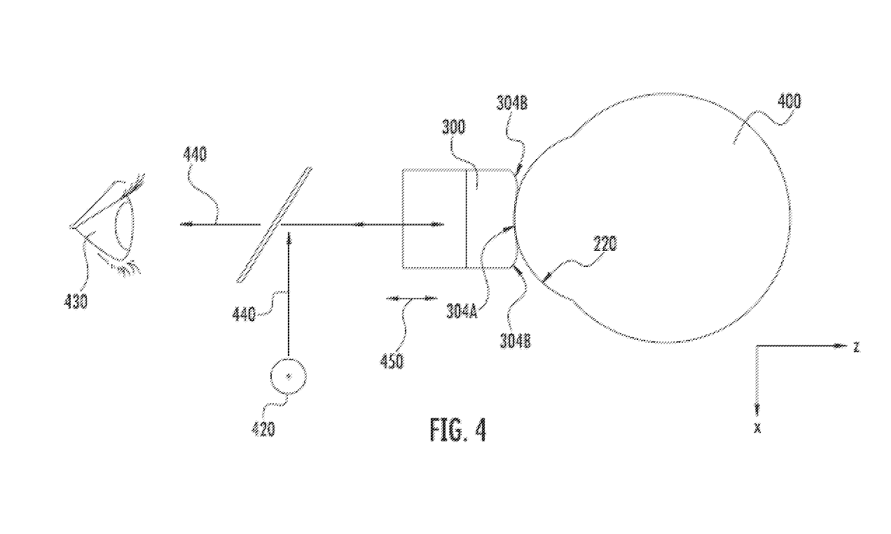

Below, and in reference to FIGS. 3A, 3B, 3C and 5A, 5B, non-limiting specific examples of the tonometer tip, shaped according to the idea of the invention, are discussed.

### EXAMPLE I

As shown in FIGS. 3A and 3B, for example, a relevant portion 300 representing, for example, a tip of an embodiment of an optical element designed to be brought in contact with the cornea of an eye (and referred to as corneal contact member), is shown in a partial cross-sectional view and a front view, respectively. A corneal contact surface 304 includes a central concave surface portion 304A, which in one specific implementation is adapted to and is preferably substantially congruent with the curvature of the cornea of a typical eye (the radius of which is approximately in the range of 7.8 mm+/−0.38 mm; the typical modulus of elasticity and range of corneal thickness for a cornea of a typical eye is discussed elsewhere in this application). The term congruent, when used in reference to two elements, specifies that these elements coincide at all points when superimposed. For the purposes of this disclosure, two surfaces are considered to be “substantially congruent” if, when superimposed, they coincide within at least 90 percent of their surface area.

At a periphery of the corneal contact surface 304, the central concave surface portion 304a passes over into and merges with, in a tangentially-parallel fashion, a peripheral surface portion 304B that has a curvature of an opposite sign (as compared to that of the central surface portion 304A). As shown in the cross-sectional view of FIG. 3A, the surface portion 304B can be characterized as convex. The peripheral surface portion 304B may define a looped (and in the specific depicted case—annular) projection along the axis 306 and onto a plane transverse to the axis 306, and forms an annulus, a ring around the central portion 304A. The central concave surface portion 304A and the peripheral annular portion 304B tangentially and seamlessly merge into each other along a closed curve 310 defined in a plane that is tangential to the surface 304 and that extends transversely to and across the axis 306. Put differently, a first plane (which is tangential to the central surface portion 304A at the boundary 310 between the surface portions 304A, 304B) and a second plane (which is tangential to the peripheral surface portion 304B at the boundary 310 that is shared by the surface portions 304A, 304B) substantially coincide with one another and do not form a dihedral angle. The curvature of the surface 304 at any point along the curve 310 is zero.

In operation, the central concave surface portion 304A may be brought in contact with the corneal surface 220. Generally, it is not required that the tonometer tip along lateral boundary or perimeter 320 of the surface 304 meet any particular optical, mechanical, or geometrical requirement as this boundary is outside of the contact area with the cornea.

While both the perimeter curve 320 of the front surface 304 of the device 300 and the closed curve 310, along which the central curved surface portion 304A and the peripheral curved surface portion 304B are merging, are shown as circles, it is appreciated that the surface 304 can be configured such as to define at least one of these curves 310, 320 as an general ellipse (defined by the locus of points the sum of distances from which to the two given points is constant). In a specific case, however, the surface 304 is rotationally symmetric about an axis 306. The example of FIGS. 3A and 3B shows just such rotationally symmetric surface 304.

In one implementation, and in further reference to FIGS. 3A, 3B, the concave surface portion 304A includes a spherical surface having a radius of curvature R of e.g. about −9.0 mm (defined in a plane containing the axis 306), and a footprint or normal projection along the axis 306 with a diameter d of e.g. about 3.06 mm (defined in a plane transverse to the axis 306). The peripheral annular (i.e., having a form of a ring) surface portion 304B has a radius of curvature of e.g. about 3.0 mm (defined in a plane containing the axis 306). In such implementation, the footprint or projection of the corneal contact surface 304 onto the plane normal to the axis 306 defines a circle with a diameter D of e.g. about 6.0 mm. The corneal contact surface 304 may be formed in a polymeric material (for example, polycarbonate, with a refractive index on the order of 1.5) or glass with polished finish of optical quality.

### EXAMPLE II

In an embodiment related to the embodiment 300 of FIGS. 3A, 3B, the corneal contact surface 304 is modified, as compared with the embodiment 300, such as to have different extents in different directions and, generally, a non-axially-symmetric footprint or normal projection. In such a case, the central concave surface portion of the corneal contact surface, while remaining substantially fitted (curvature wise) to the corneal surface, may have unequal extents in two (in a specific case—mutually perpendicular) directions. Accordingly, the peripheral surface portion, while remaining adjoining to the central concave surface portion in a fashion described above, also has a ratio of lateral extents that is similar or even equal to the ratio characterizing the central concave portion.

In a specific example shown in top view in FIG. 3C, the so-configured corneal contact surface 350 has footprint 352 defined by an ellipse or oval on a plane that is perpendicular to the z-axis. The surface 350 includes a central, substantially spherical surface portion 354A and a peripheral annular portion 354B, each of which has an elliptically-shaped corresponding projection on the plane that is perpendicular to the axis 306 (which, in FIG. 3C, is parallel to the axis z of the indicated local system of coordinates). As shown, the dimensions of the central surface portion 354A along the minor and major axes of the corresponding footprint are a and b, respectively. The maximum dimensions of the peripheral surface portion 354B along the corresponding minor and major axes of its footprint are A and B, respectively, and indicated by a perimeter 320′. The surface portions 354A, 354B are tangentially, seamlessly merging into one another along an elliptical closed plane curve 310′ in a fashion similar to that described in reference to FIGS. 3A and 3B. In this specific example, the corneal contact surface is axially symmetric. In one implementation, a is about 2.13 mm, b is about 3.06 mm. The bi-prismatic element (not shown) that is internal to the corneal contact member having the surface 350 may be oriented such as to approximately bisect the long extent B of the footprint 352 of FIG. 3C.

The implementation illustrated in FIG. 3C is adapted to facilitate the measurements of the IOP of the patients with interpalpebral features that may not necessarily allow the observer-examiner to accommodate a symmetrically-structured corneal contact surface of the embodiment of FIGS. 3A and 3B. It is appreciated that, when the implementation of the invention the operation of which is represented by FIG. 3C is used in practice, the area of the cornea subject to applanation remains substantially the same as that corresponding to the embodiment of FIG. 3B. The lateral dimension of the oval footprint corresponding to 354A that accommodates a narrow interpalpebral fissure (partially closed lids) is reduced, while the orthogonal dimension of the footprint (along the eye lids) is increased, as compared to the diameter of the footprint 304A. Under some conditions, the force required to achieve applanation may be reduced.

Generally, a cornea-contacting surface of the corneal contact member 300 is structured to include an azimuthally symmetric bi-curved surface having a cross-section that is defined (in a plane containing an optical axis of the contact member 300) by an axially-symmetric monotonic curve having first and second local maxima; one minimum that coincides with the axis of symmetry of such curve; and a second derivative defined at any point of such axially-symmetric monotonic curve. Such cornea contact surface includes a central concave portion and a peripheral convex portion that circumscribes the central concave portion. In operation, the central concave portion of the corneal contact surface produces a substantially negligible compression of the central portion of the cornea with which it comes in contact. A region of the corneal contact surface along which the peripheral convex portion and the central contact portion adjoin each other produces a slight corneal compression to define a peripheral ring pattern, observed in form of semicircles, in reflection of light from the cornea.

### EXAMPLE III

FIGS. 5A, 5B schematically depict a related embodiment 500 of a tip of the corneal contact member shown in a partial cross-sectional view and a front view, respectively. A corneal contact surface 504 includes a central surface portion 504A, the curvature of which has a sign opposite to the sign of the curvature of the cornea. At a periphery of the corneal contact surface 504, the central surface portion 504A passes over into and tangentially merges with a peripheral surface portion 504B that has a curvature of an opposite sign (as compared to that of the central surface portion 504A). As shown in the cross-sectional view of FIG. 5A, the surface portion 504A can be characterized as convex. The peripheral concave surface portion 504B defines a looped (and in the specific case—annular) projection along the axis 506 and onto a plane transverse to the axis 506. The central convex surface portion 504A and the peripheral concave annular portion 504B are tangentially, seamlessly merging into each other along a closed curve 510 defined in a plane that is both tangential to the surface 504 and transverse to the axis 506. Put differently, a first plane (which is tangential to the central surface portion 504A at the boundary 510 between the surface portions 504A, 504B) and a second plane (which is tangential to the peripheral surface portion 504B at the boundary 510 that is shared by the surface portions 504A, 504B) substantially coincide with one another and do not form a dihedral angle. The curvature of the surface 504 at any point along the curve 510 is substantially zero.

In operation, the central convex surface portion 504A is brought in contact with the corneal surface 220. Generally, it is not required that the tonometer tip along lateral boundary or perimeter 520 of the surface 504 meet any particular optical, mechanical, or geometrical requirement as this boundary is outside of the contact area with the cornea.

While both the perimeter curve 520 of the front surface 504 of the device 500 and the closed curve 510, along which the central curved surface portion 504A and the peripheral curved surface portion 504B are merging, are shown as circles, it is appreciated that the surface 504 can be configured such as to define at least one of these curves 510, 520 as an general ellipse. In a specific case, however, the surface 504 is rotationally symmetric about an axis 506. The example of FIGS. 3A and 3B shows just such rotationally symmetric surface 504.

In one implementation, and in further reference to the embodiment of FIGS. 5A, 5B, the convex surface portion 504A includes a spherical surface having a radius of curvature R of about +9.0 mm (defined in a plane containing the axis 506), and a footprint or normal projection along the axis 506 with a diameter d of about 3.06 mm (defined in a plane perpendicular to the axis 506). The peripheral annular (i.e., having a form of a ring) surface portion 504B has a radius of curvature of about 3.0 mm (defined in a plane containing the axis 506). In such implementation, the footprint or projection of the corneal contact surface 504 onto the plane normal to the axis 506 defines a circle with a diameter D of about 3.06 mm. The corneal contact surface 504 may be formed in a polymeric material (for example, polycarbonate, with a refractive index on the order of 1.5) or glass with polished finish of substantially optical quality. A lateral boundary or perimeter 520 of the surface 504 may not be required to meet any particular optical, mechanical, or geometrical requirement as it is outside of the contact area with the cornea.

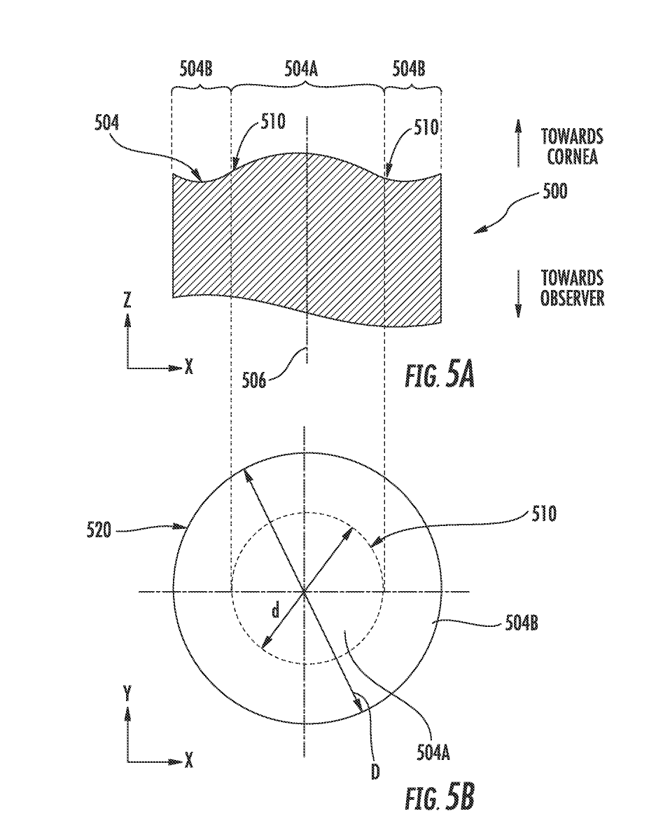

A related implementation 600 of the tonometer tip, having a corneal contact surface 504, is schematically shown in a partial cross-sectional view of FIG. 6. As shown, the radius, defined with respect to the 506, at which the annular concave portion 504B reaches its lowest point (an extremum) 604 is 1.15 mm; the axial separation between the apex 608 of the tip 600 and the peripheral edge 510 is 29 microns; the axis separation between the apex 608 of the portion 504A and the bottom 604 of the portion 504B is 60 microns; and the overall radius of the tip, measured in a plane that is perpendicular to the axis 506, is 1.505 mm.

The profile of the surface 504 of the embodiment 600 was determined by optimizing a general surface 504, represented with a polynomial, such as to minimize the second derivative of the profile of the cornea with which the embodiment 600 is brought in forceful contact. The optimization was carried out by minimizing the modulus of the von Mises stress averaged, at a given radius, through the thickness of the cornea.

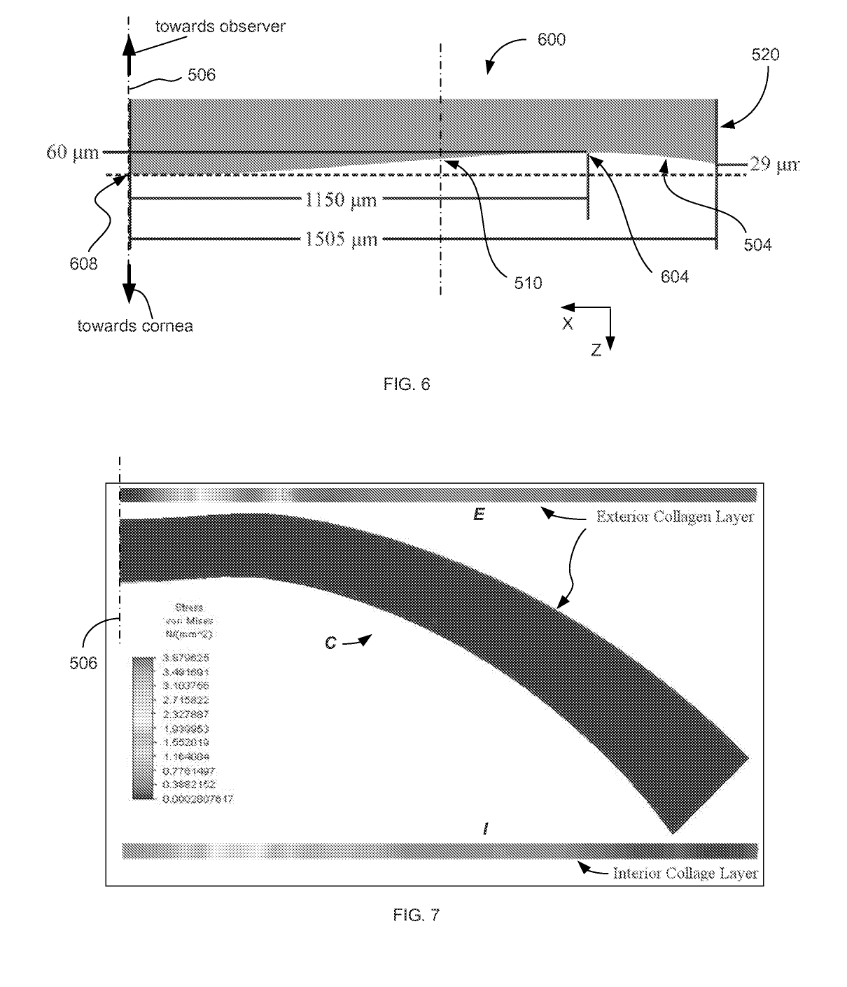

The polynomial optimization of the corneal contact surface 504 of the embodiment 500 was performed with the use of a finite-element method for an average, typical cornea (having an external radius of curvature of about 7.8 mm and an average corneal modulus of elasticity of 0.58 MPa). FIG. 7 illustrates, in partial cross-sectional view, the average cornea C with indication of spatial distribution of stress formed in the exterior collagen layer E (at the exterior surface of the cornea) and those in the interior collagen layer I (at the interior surface of the cornea). The term “average cornea” refers to a cornea with geometrical and mechanical parameters that are averaged based on known statistical distribution of such cornea parameters across population, i.e. that represented by statistical average of geometric and material properties of human corneas.

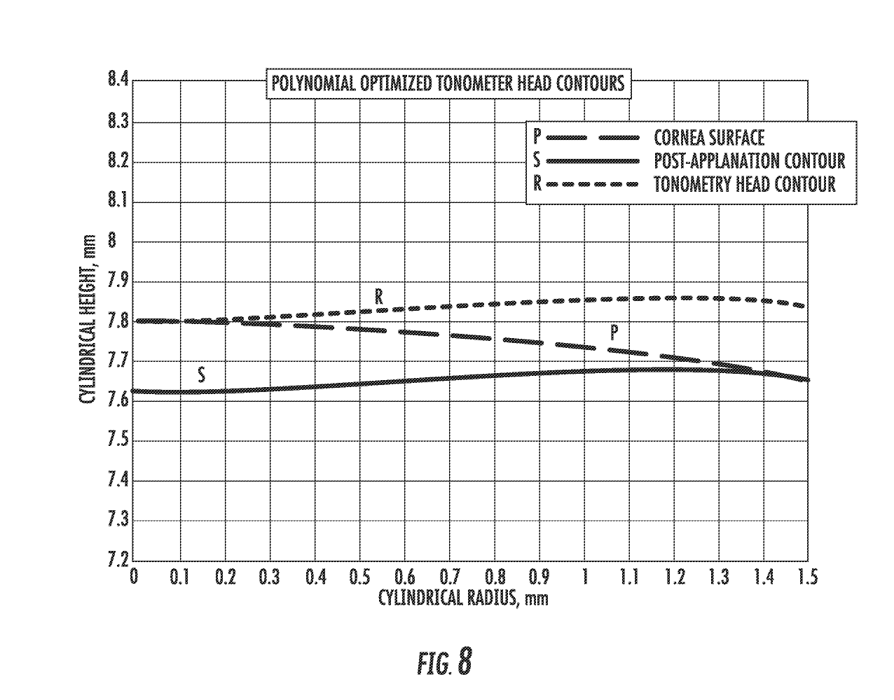

The degree to which the profile of the average cornea changes when it is brought in contact with the surface 504 of the embodiment 600, illustrated with the use of a polynomial fitting, is shown in FIG. 8 that provides a comparison, on the same spatial scale, the radial profile P of the surface of the free-standing (not in contact with any external tool) cornea, the radial profile R of the surface 504 of the embodiment 600 of the instrument, and the radial profile S of the same cornea post-applanation with the embodiment 600 that is brought in contact with the cornea. The zero value along the y-axis (“cylindrical height”) corresponds to the center of corneal curvature.

### EXAMPLE IV

In an embodiment (not shown), the corneal contact surface 504 can be modified such as to have at least one of the perimeter 520 and the curve 510 define a general ellipse. The annular portion 504B could also be shaped to define a corresponding elliptically-shaped ring around the central convex surface portion 504A.

To illustrate the operational advantage of the tonometer tip configured according to an idea of the invention, the shape of the cornea-contacting surface of the tip of the device of the invention can also be assessed within ranges of several parameters that cause the error in measuring the IOP. Among such parameters are a corneal curvature (6-9 mm 95%; 6 mm being a curvature of a very steep cornea), and corneal modulus of elasticity (0.1-0.9 MPa 95%; 0.9 MPa being a modulus of a very rigid cornea), thickness of the cornea (450-700 microns 95%), and thickness of tear film (0-1 mm 95%)

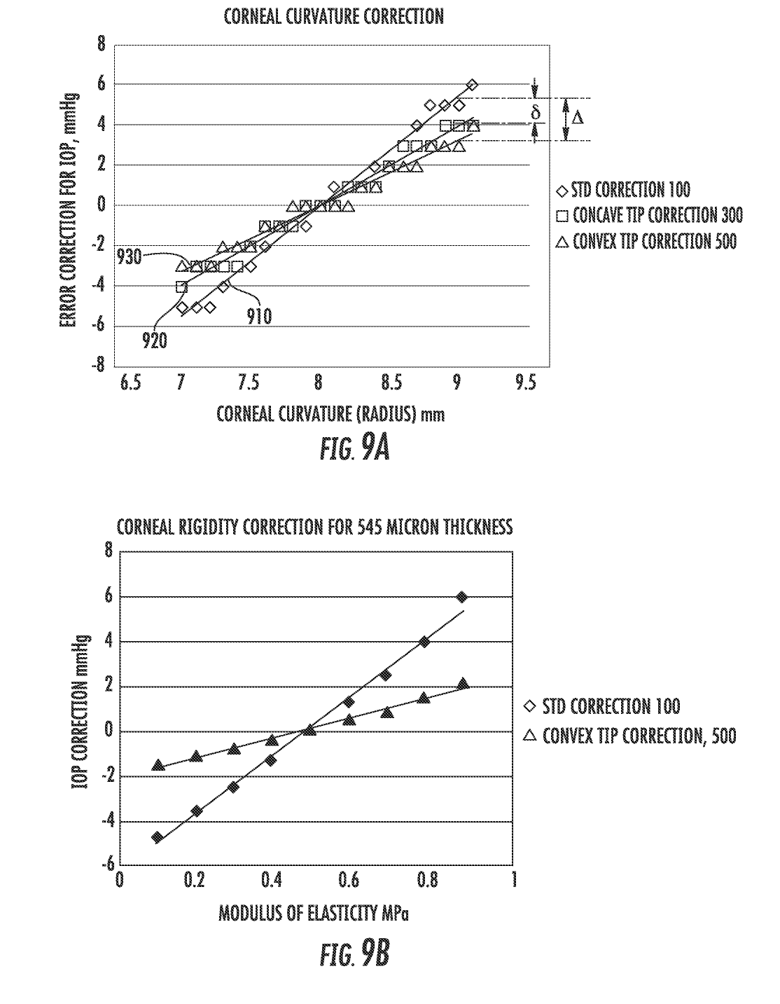

Reduction of a Measurement Error due to Corneal Curvature, Caused by the Use of an Embodiment of the Invention. The calculated with the use of the finite-element method (FEM) value of correction for intraocular pressure, required to be taken into account due to the presence of the corneal curvature, is presented in FIG. 9A for each of a conventional flat-tip corneal contact member 100 (data and linear fit 910), the embodiment 300 of the present invention (data and linear fit 920), and the embodiment 500 of the present invention (data and linear fit 930). The radius of corneal curvature was varied from 6.8 to 9.4 mm, to accommodate empirically known deviations of corneal curvatures from that of an averaged, standard corneal curvature. A skilled artisan would appreciate that the measurements of the IOP carried out with a tonometer tip dimensioned according to an embodiment of the invention (such as 300 or 500) imposes smaller intraocular stress on the cornea as compared with those performed with a flat-tip tonometer and, consequently, the contribution of error caused by the corneal curvature to the results of the measurement is smaller for the embodiments 300, 500. For example (and considering a particular cornea having a 9 mm radius), the correction to the IOP that has to be introduced to take into account the corneal curvature when the measurement is performed with the embodiment 300 is by δ≈1 mmHg or more smaller than the correction required when the flat-tip corneal contact member 100 is used. The use of the embodiment 500 results in an even more precise measurements: here, the error introduced by the corneal curvature is by Δ≈2 mmHg (or even more) smaller that the corresponding error accompanying the measurement with the embodiment 100. Clearly, improving the achievable accuracy of determination of the IOP by about 2 mmHg (out of the standard 16 mmHg of intraocular pressure, or by more than 12%) makes a practical difference in the determination of whether a particular eye has to be operated on. While the influence of the presence of the tear film is expected to somewhat affect the results of the IOP measurements, it was not included in the model.

Reduction of a Measurement Error due Corneal Rigidity, Caused by the Use of an Embodiment of the Invention. While addressing the influence modulus of elasticity of the composite material of the cornea on the IOP measurement error, on the other hand, the empirically known range of such modulus from about 0.1 MPa to about 0.9 MPa has to be taken into account. FIG. 9B provides plots illustrating that correction to the measured IOP value (required to compensate for the error caused by the corneal rigidity) is substantially reduced when the cornea-contacting surface of the tonometer tip is structured according to the idea of the embodiment 500. The calculations were performed with the FEM for a cornea with thickness of 545 microns (which provides a mid-value for the practically common range of corneal thickness, for a typical cornea, from about 475 microns to about 640 microns). For known individual variations of corneal rigidity, the use of the tonometer tip that is optimized by being configured according to the principles in the examples described above (as compared with the conventional standard of the flat tip) reduces the error by as much as 2 mmHg.

Reduction of a Measurement Error due Corneal Thickness, Caused by the Use of an Embodiment of the Invention. Plots of FIG. 9C illustrate the results of clinical comparison in vivo of the errors introduced to the IOP measurement by the embodiments 100 and 500 of the tonometer tip. A clear trend could be observed towards substantial reduction of error when the measurement of the IOP is performed with the tonometer tip configured according to the idea of the invention. The practically observed reduction in error, attributed to the non-zero corneal thickness, of up to 2 mmHg—as defined by the use of a tonometer tip configured in accord with the idea(s) of the present invention, and as compared with that during the measurements performed with the conventional flat-surface tonometer tip—is in line with the predictions made by the mathematical model (linear fit).

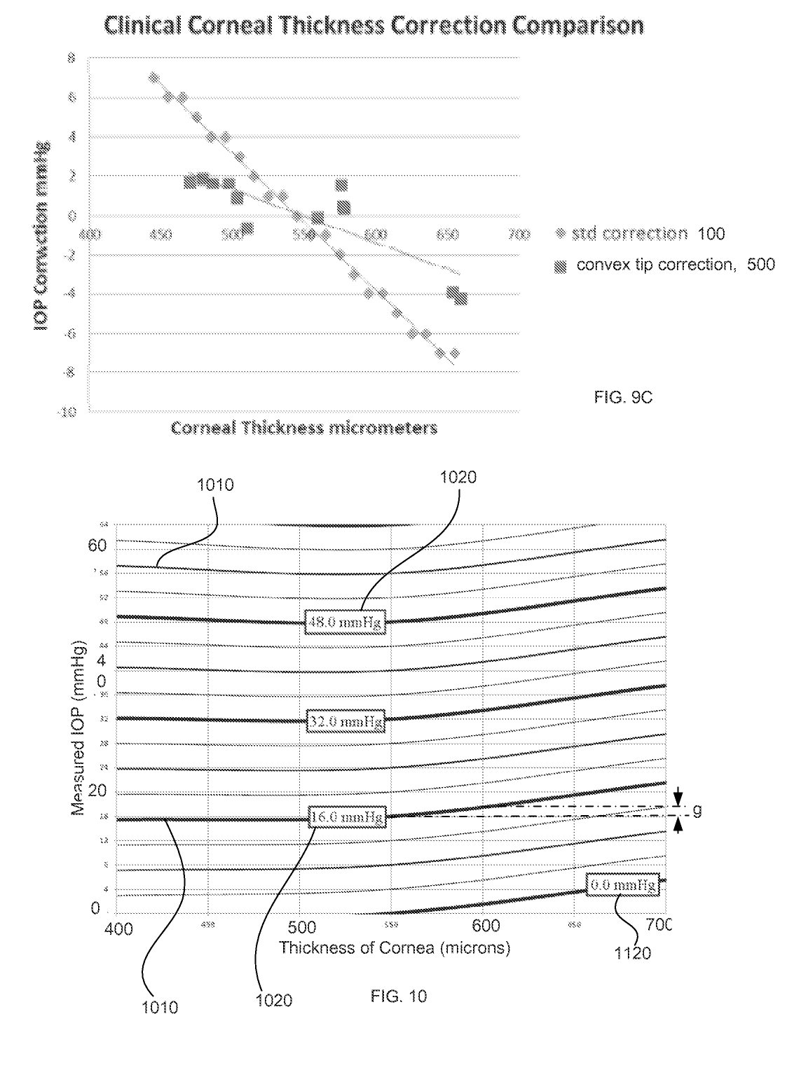

FIG. 10, showing the isobaric curves devised with the use of the FEM for the standard cornea, further facilitates the assessment of influence of the thickness of the standard cornea on the value of measured IOP (isobaric curves 1010) in comparison with the actual IOP (shown as values in blocks 1020). For example, for a typical IOP of about 16 mmHg, the measured value of the IOP will exceed the actual IOP due to the error of about 1.5 mmHg to 2.0 mmHg.

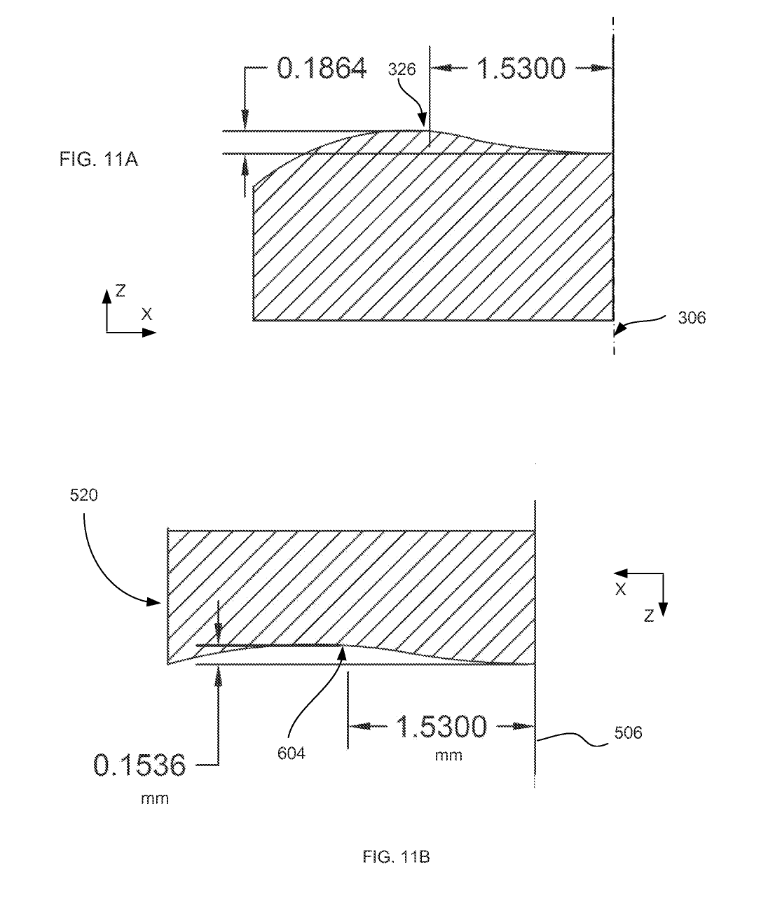

Worth noting is the practical possibility of extreme eye-characteristics that contribute maximally to the measurement error in Goldmann applanation tonometry. Such characteristics include a steep cornea of 6 mm radius, a rigid cornea 0.9 MPa, a cornea with the central thickness of 700 microns, and zero tear film. To this end, FIG. 11A provides parameters of a specific design of the rotationally-symmetric version of surface 304 devised for such extreme situation. As shown, the radius (defined with respect to the axis 306) at which the annular convex portion 304B reaches its top point (an extremum, apex) 326 is 1.53 mm; and the axial separation between the apex of the peripheral portion 304B and the center of the surface 304 (the point of surface 304 at the axis 306) is about 186 microns. Similarly, FIG. 11B provides parameters of a specific design of the surface 504 devised for such extreme situation. Therefore, the judiciously defined curved/non-flat configuration of a cornea-contacting surface of a tonometer tip allows to reduce measurement errors attributed to the biomechanical properties of the eye not only for the typical eye with standard characteristics but also for an eye with rare, extreme characteristics.

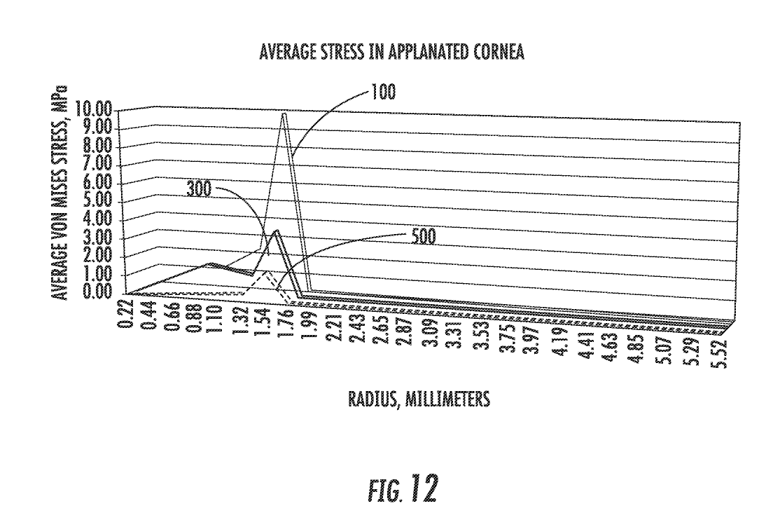

It is appreciated from the above discussion that the key to devising an optimized tonometer tip is minimization of intracorneal stress during the applanating deformation occurring during the IOP measurement. FIG. 12 illustrates additional guidance to advantages provided by the embodiments 300 and 500 of the invention in comparison with the currently used flat-tip standard of the GAT. Shown is the average intracorneal stress (von Mises stress) at a given applanated radial distance from the corneal apex. The use of the tonometer tips dimensioned according to idea of the present invention reduced intraocular stress, and also reduces the second derivative of the deformed corneal surface (or the rate of change of the corneal curvature).

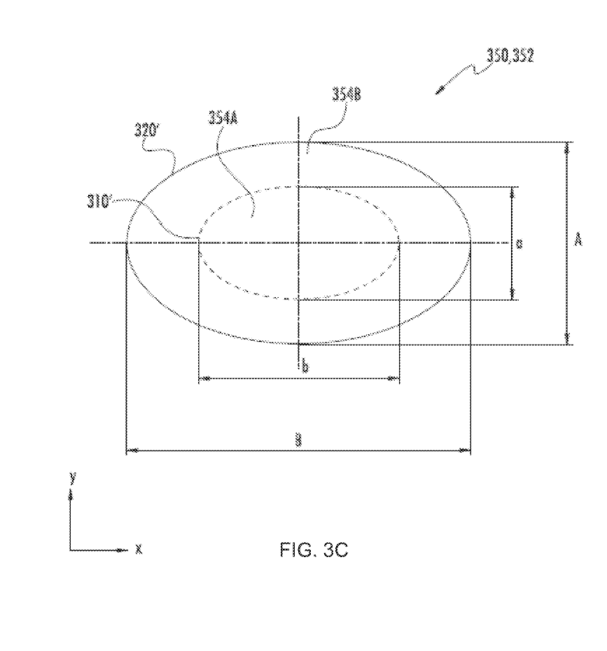

A schematic diagram of FIG. 4 illustrates a process of the examination of an eye 400 with a tonometer a tip of which is configured according to the embodiment 300 of FIGS. 3A, 3B. (A similar process of examination would be carried out with the embodiment 500). During the measurement of the IOP, the corneal contact member 300 (having the surface 304 or the surface 350) is brought in contact with the corneal surface 220. The cornea-contacting surface 304 (or surface 350), of the member 300 is shaped according to a corresponding embodiment of the invention and dimensioned to minimize the deformation of the corneal surface 220 during the IOP-measurement procedure with the use of a Goldmann tonometer. In particular, and as will be understood by a skilled artisan, the minimization of the corneal deformation translates to minimization of the contribution of the corneal stiffness into the force defined by the eye in response to the applied measurement of the force (that, in turn, is required for proper applanation of a portion of the corneal surface that defines a circular area with a diameter of about 3.06 mm). As a practical result of such reduction or minimization of the corneal contribution, the correction factor (which takes into account corneal thickness and that is used to practically unreliable compensate for the unknown corneal stiffness, as discussed above) becomes substantially negligible. The computational compensation of the errors of the measurement of the IPO, therefore, becomes practically unnecessary. Similarly, a need to perform costly and time-consuming pachymetries, directed to correcting a cornea-thickness-related error that accompanies conventionally performed measurements of the IPO with the use of the Goldmann tonometer, is substantially eliminated, thereby leading to a measurement method that does not include pachymetry.

In further reference to FIG. 4, some components of the GAT are omitted for the simplicity of illustration. The path of light, traversing the bi-prism-containing corneal contact member 300 on its propagation from a light source 420, to a reflecting element 424, to the surface 220 of the cornea (and, in reflection, to an observer 430) is designated with arrows 440. A variable pressure force, applied to the corneal surface 220 is designated with an arrow 450.

It is understood that specific numerical values, chosen for illustration of examples of embodiments described in reference to FIGS. 3A, 3B, and 4, may generally vary over wide ranges to suit different applications. It will be understood by those of ordinary skill in the art that modifications to, and variations of, the illustrated embodiments may be made without departing from the inventive concepts disclosed herein. Both of the central concave surface portion and the associated peripheral surface portion of the corneal contact surface may be uninterrupted and spatially continuous (such as the portions 304A, 304B of FIGS. 3A, 3B or the portions 354A, 354B of FIG. 3C, for example). Alternatively, at least one of the central concave portion and the associate peripheral surface portion may be spatially discontinuous (at least in one direction transverse to the optical axis of the corneal contact member) such as to define, in a projection onto a plane perpendicular to the optical axis of the corneal contact member, a segmented footprint of the corneal contact surface. For example, at least one of the central concave surface portion and the peripheral surface portion may be spatially interrupted such as to preserve symmetry of such interrupted surface portion(s) with respect to at least one spatial axis. In reference to FIGS. 3A, 3B, and as a specific example, the peripheral surface portion 304B may be spatially interrupted along the y-axis. In operation, when pressed against the cornea, such segmented structure will define a plurality of applanation areas that are located substantially symmetrically about an axis along which the surface interruption is present (in this case, along the y-axis).

Overall, the use of a tonometer tip the corneal-contacting surface of which is formatted to deviate from the flat, planar surface and configured as including a curved surface having two having curvatures of opposite signs, as described above, have been demonstrated to increase the accuracy of the IOP measurement over those performed with the conventionally-used GAT that employs the tonometer tip with the flat surface and to at least reduce a need in and value of correction of the results of the measurement to take into account at least one of the central corneal thickness (or CCT), corneal rigidity or stiffness, corneal curvature, and/or intracorneal stress.

The invention as recited in claims appended to this disclosure is intended to be assessed in light of the disclosure as a whole, including features disclosed in prior art to which reference is made. Accordingly, the invention should not be viewed as being limited to the disclosed embodiment(s).

What is claimed is:

1.-21. (canceled)

22. An optical instrument f©r measurement of intraocular pressure (IOP) of an eye, the instrument comprising:
a corneal contact member with a front surface that is dimensioned to contact the cornea of the eye during the measurement, said front surface including
(i) a corneal contact surface portion defined by a central portion of the front surface of the corneal contact member, said corneal contact surface portion being curved with non-zero curvature and being non-flat to reduce an error contributed to said measurement by at least a curvature of the cornea; and
(ii) a peripheral surface portion surrounding a curved corneal contact surface portion and tangentially merging with said corneal contact surface portion along a closed plane curve,
wherein, when the cornea of the eye has a first curvature with a first sign, the corneal contact surface portion has a second curvature with a second sign opposite to the first sign, and the peripheral surface portion has a third curvature with a third sign, said third sign being opposite to the second sign.

23. An instrument according to claim 22, wherein the corneal contact member has a longitudinal axis, and wherein said closed plane curve is defined in a plane that is transverse to said longitudinal axis.

24. An instrument according to claim 22, wherein the corneal member has a longitudinal axis, and wherein said front surface is shaped to change a sign of the first curvature within a surface area centered on the longitudinal axis.

25. An instrument according to claim 22, wherein said front surface is shaped to flatten a portion of the cornea when said corneal contact member is pressed, in operation, against the cornea, said flattened portion of the cornea defined by a surface area symmetric about the longitudinal axis.

26. An instrument according to claim 22, further comprising
an optical prism in a body of the corneal contact member, and
a source of light positioned to transmit light through the prism towards the front surface.

27. An instrument according to claim 22, wherein the corneal contact surface portion defines a portion of a spherical surface, said corneal contact surface portion being devoid of openings therethrough.

28. An instrument according to claim 22, wherein the corneal contact member has a longitudinal axis and the front surface is axially symmetric about said longitudinal axis.

29. An instrument according to claim configured as a tonometer.

30. An instrument according to claim 22, further comprising a housing element having an outer conical surface, said corneal contact member being fixed in said housing.

31. An instrument according to claim 22, wherein said front surface is dimensioned to minimize intracorneal stress in said applanated portion of the cornea.

32. An optical instrument for measurement of intraocular pressure (IOP), the instrument comprising:
a corneal contact member having a front surface that is dimensioned to contact a first portion of the cornea of an eye, said front surface being rotationally symmetric about a longitudinal axis and including
i) a corneal contact surface portion defining a portion of a spherical surface devoid of openings therethrough, said corneal contact surface portion having a first curvature with a first sign opposite to a sign of a curvature of the cornea; and
(ii) a peripheral surface portion surrounding the corneal contact surface portion and tangentially merging with said corneal contact surface portion along a closed curve defined in a plane that is transverse to said longitudinal axis, the peripheral surface portion having a second curvature, the second curvature having a second sign that is equal to a sign of the curvature of the cornea.

33. An instrument according to claim 32, wherein said front surface is shaped to applanate a portion of the cornea to form an applanated portion of the cornea, when said corneal contact member is pressed, in operation, against the cornea.

34. An instrument according to claim 32, wherein said front surface is dimensioned to minimize intracorneal stress in said applanated portion of the cornea.

35. An instrument according to claim 32, further comprising an optical prism in a body of the conical contact member and a source of light positioned to transmit light through the prism towards the front surface.

36. An instrument according to claim 32, wherein said front surface is shaped to change a sign of the first curvature within a surface area centered on the longitudinal axis.

37. An instrument according to claim 32, wherein the corneal contact surface portion defines a portion of a spherical surface, said cortical contact surface portion being devoid of openings therethrough.

38. An instrument according to claim 32 configured as a tonometer.

39. An instrument according to claim 32, further comprising a housing element having an outer conical surface, said corneal contact member being fixed in said housing element.

40. A method for measuring intraocular pressure (IOP) with an optical instrument, the optical instrument including a corneal contact member, said corneal contact member having a corneal contact surface that defines
(i) a central curved portion having a surface curvature of a first sign, and
(ii) a peripheral surface portion having a surface curvature of a second sign, wherein the second sign is opposite to the first sign, and wherein said peripheral surface portion surrounds the central curved portion the method comprising:
pressing said corneal contact member against the cornea to establish a contact between the conical contact surface and the cornea and to applanate a first portion of the cornea while minimizing an error contributed to said measuring by a curvature of the cornea, wherein the curvature of the cornea has a third sign, the third sign being opposite to the first sign;
forming an optical image of the cornea in light traversing said corneal contact member and the corneal contact; and
determining a value of the IOP from imaging data representing said optical image.

41. A method according claim 40, that is devoid of a step of correcting the imaging data to compensate for error contributed to said measuring by at least one of corneal thickness and corneal stiffness.

42. A method according to claim 40, wherein said pressing includes pressing said corneal contact member in which the peripheral surface portion is tangentially merging with the central curved portion along a closed plane curve.
